(19) United States
(12) Patent Application Publication
(10) Pub. No.: US 2019/0315846 A1
(43) Pub. Date: Oct. 17, 2019

(54) Antibodies to Human Alpha-Synuclein

(71) Applicant: THE MICHAEL J. FOX FOUNDATION FOR PARKINSON'S RESEARCH, New York, NY (US)

(72) Inventors: Terina N. Martinez, Brooklyn, NY (US); Kuldip Dave, Spotswood, NJ (US); Sonal Das, Cambridge, MA (US)

(73) Assignee: THE MICHAEL J. FOX FOUNDATION FOR PARKINSON'S RESEARCH, New York, NY (US)

(21) Appl. No.: 16/466,713

(22) PCT Filed: Dec. 7, 2017
(86) PCT No.: PCT/US2017/065035

§ 371 Date: Jun. 5, 2019

Related U.S. Application Data
(60) Provisional application No. 62/432,761, filed on Dec. 12, 2016.

(51) Int. Cl. C07K 16/18; A61K 47/68

(52) U.S. Cl. CPC C07K 16/18; A61K 47/6803; C07K 2317/569; C07K 2317/24; C07K 2317/34

(57) ABSTRACT

Described a monoclonal antibody to human alpha-synuclein, and the use of that antibody in treating Parkinson's disease.

### FIELD

This disclosure concerns monoclonal antibodies, such as single-domain monoclonal antibodies, specific for α-synuclein. This disclosure further concerns the use of such antibodies, such as for the detection and treatment of Parkinson's Disease.

### BACKGROUND

Alpha-synuclein is a protein that is abundant in the human brain. Smaller amounts are found in the heart, muscles, and other tissues. In the brain, alpha-synuclein is found mainly at the tips of nerve cells (neurons) in specialized structures called presynaptic terminals. Within these structures, alpha-synuclein interacts with phospholipids and proteins. Presynaptic terminals release chemical messengers, called neurotransmitters, from compartments known as synaptic vesicles. The release of neurotransmitters relays signals between neurons and is critical for normal brain function.

Although the function of alpha-synuclein is not well understood, studies suggest that it plays a role in maintaining a supply of synaptic vesicles in presynaptic terminals by clustering synaptic vesicles. It may also help regulate the release of dopamine, a type of neurotransmitter that is critical for controlling the start and stop of voluntary and involuntary movements.

The human alpha-synuclein protein is made of 140 amino acids and is encoded by the SNCA gene. An alpha-synuclein fragment, known as the non-Abeta component (NAC) of Alzheimer's disease amyloid, originally found in an amyloid-enriched fraction, was shown to be a fragment of its precursor protein, NACP. It was later determined that NACP was the human homologue of Torpedo synuclein. Therefore, NACP is now referred to as human alpha-synuclein.

### Tissue Expression

Alpha-synuclein makes up as much as 1% of all proteins in the cytosol of brain cells. lis predominantly expressed in the neocortex, hippocampus, substantia nigra, thalamus, and cerebellum. It is predominantly a neuronal protein, but can also be found in the neuroglial cells. In melanocytic cells, SNCA protein expression may be regulated by MITF.

It has been established that alpha-synuclein is extensively localized in the nucleus of mammalian brain neurons, suggesting a role of alpha-synuclein in the nucleus. Synuclein is however found predominantly in the presynaptic termini, in both free or membrane-bound forms, with roughly 15% of synuclein being membrane-bound in any moment in neurons.

Recently, it has been shown that alpha-synuclein is localized in neuronal mitochondria. Alpha-synuclein is highly expressed in the mitochondria in olfactory bulb, hippocampus, striatum and thalamus, where the cytosolic alpha-synuclein is also rich. However, the cerebral cortex and cerebellum are two exceptions, which contain rich cytosolic alpha-synuclein but very low levels of mitochondrial alpha-synuclein. It has been shown that alpha-synuclein is localized in the inner membrane of mitochondria, and that the inhibitory effect of alpha-synuclein on complex I activity of mitochondrial respiratory chain is dose-dependent. Thus, it is suggested that alpha-synuclein in mitochondria is differentially expressed in different brain regions and the background levels of mitochondrial alpha-synuclein may be a potential factor affecting mitochondrial function and predisposing some neurons to degeneration.

At least three isoforms of synuclein are produced through alternative splicing. The majority form of the protein, and the one most investigated, is the full-length protein of 140 amino acids. Other isoforms are alpha-synuclein-126, which lacks residues 41-54 due to loss of exon 3; and alpha-synuclein-112, which lacks residue 103-130 due to loss of exon 5.

### Clinical Significance

Alpha-synuclein aggregates to form insoluble fibrils in pathological conditions characterized by Lewy bodies, such as Parkinson's disease, dementia with Lewy bodies and multiple system atrophy. These disorders are known as synucleinopathies. Alpha-synuclein is the primary structural component of Lewy body fibrils. Occasionally, Lewy bodies contain tau protein; however, alpha-synuclein and tau constitute two distinctive subsets of filaments in the same inclusion bodies. Alpha-synuclein pathology is also found in both sporadic and familial cases with Alzheimer's disease.

The aggregation mechanism of alpha-synuclein is uncertain. There is evidence of a structured intermediate rich in beta structure that can be the precursor of aggregation and, ultimately, Lewy bodies. A single molecule study in 2008 suggests alpha-synuclein exists as a mix of unstructured, alpha-helix, and beta-sheet-rich conformers in equilibrium. Mutations or buffer conditions known to improve aggregation strongly increase the population of the beta conformer, thus suggesting this could be a conformation related to pathogenic aggregation. Among the strategies for treating synucleinopathies are compounds that inhibit aggregation of alpha-synuclein. It has been shown that the small molecule cuminaldehyde inhibits fibrillation of alpha-synuclein. The Epstein-Barr virus has been implicated in these disorders.

In rare cases of familial forms of Parkinson's disease, there is a mutation in the gene coding for alpha-synuclein. Five point mutations have been identified thus far: A53T, A30P, E46K, H50Q, and G51D. Genomic duplication and triplication of the gene appear to be a rare cause of Parkinson's disease in other lineages, although more common than point mutations. Hence certain mutations of alpha-synuclein may cause it to form amyloid-like fibrils that contribute to Parkinson's disease.

Certain sections of the alpha-synuclein protein may play a role in the tauopathies.

### SUMMARY

Disclosed herein are α-synuclein-specific antibodies. The antibodies bind specifically to human α-synuclein. The antibodies provided herein include immunoglobulin molecules, such as IgG antibodies, as well as antibody fragments and single-domain (VH) antibodies. Further provided are compositions including the antibodies that bind, for example specifically bind, to α-synuclein, nucleic acid molecules encoding these antibodies, expression vectors comprising the nucleic acid molecules, and isolated host cells that express the nucleic acid molecules. Also provided are immunoconjugates comprising the antibodies disclosed herein and an effector molecule. Fusion proteins comprising the antibodies are also provided, such as fusion proteins comprising human Fc.

The antibodies and compositions provided herein can be used for a variety of purposes, such as for confirming the diagnosis of a pathological condition characterized by Lewy bodies, termed a synucleinopathy. Common synucleinopathies include Parkinson's disease, dementia with Lewy bodies, and multiple system atrophy. Thus, provided herein is a method of confirming the diagnosis of a synucleinopathy in a subject by contacting a sample from the subject diagnosed with Parkinson's disease with a monoclonal antibody that binds α-synuclein, and detecting binding of the antibody to the sample. An increase in binding of the antibody to the sample relative to binding of the antibody to a control sample confirms the diagnosis. In some embodiments, the method further includes contacting a second antibody that specifically recognizes the α-synuclein-specific antibody with the sample, and detecting binding of the second antibody.

Similarly, provided herein is a method of detecting a disorder characterized by aggregation of α-synuclein in a subject. The method includes contacting a sample from the subject with a monoclonal antibody described herein, and detecting binding of the antibody to the sample. An increase in binding of the antibody to the sample relative to a control sample detects the aggregation of α-synuclein in the subject. In some embodiments, the methods further comprise contacting a second antibody that specifically recognizes the α-synuclein-specific antibody with the sample, and detecting binding of the second antibody.

Further provided is a method of treating a subject having a pathological condition characterized by Lewy bodies, termed a synucleinopathy. The method includes selecting a subject having a synucleinopathy, and administering to the subject a therapeutically effective amount of a monoclonal antibody specific for α-synuclein, or an immunoconjugate, fusion protein or composition comprising the antibody.

The foregoing and other objects, features, and advantages of the invention will become more apparent from the following detailed description, which proceeds with reference to the accompanying figures.

### BRIEF DESCRIPTION OF THE DRAWINGS

FIG. 1 shows the α-synuclein amino acid sequence (human and mouse), and shows the hexapeptide YQDYEP corresponding to amino acid positions 133-138 in the C-terminal.

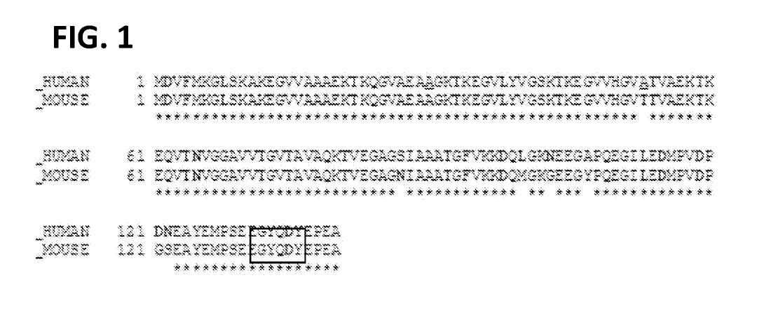

### DETAILED DESCRIPTION

### I. Abbreviations

CAR: chimeric antigen receptor

CDC: complement-dependent cytotoxicity

cDNA: complementary DNA

CDR: complementarity determining region

CTL: cytotoxic T lymphocyte

ELISA: enzyme-linked immunosorbent assay

EM: effector molecule

FACS: fluorescence activated cell sorting

GPI: glycosylphosphatidylinositol

hFc: human Fc

HRP: horseradish peroxidase

Ig: immunoglobulin

i.v.: intravenous

KD dissociation constant

LDH: lactate dehydrogenase

mAb: monoclonal antibody

MAC: membrane attack complex

NHS: normal human serum

PBMC: peripheral blood mononuclear cells

PCR: polymerase chain reaction

PE: Pseudomonas exotoxin

PE: phycoerythrin

Pfu: plaque forming units

RIPA: radioimmunoprecipitation assay

VH: variable heavy

VL: variable light

### II. Terms and Methods

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Antibody: A polypeptide ligand comprising at least a light chain or heavy chain immunoglobulin variable region which recognizes and binds (such as specifically recognizes and specifically binds) an epitope of an antigen, such as α-synuclein, or a fragment thereof. Immunoglobulin molecules are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody.

Antibodies include intact immunoglobulins and the variants and portions of antibodies well known in the art, such as single-domain antibodies (e.g. VH domain antibodies), Fab fragments, Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term “antibody” also includes genetically engineered forms such as chimeric antibodies (for example, humanized murine antibodies) and heteroconjugate antibodies (such as bispecific antibodies). See also, Pierce Catalog and Handbook, 1994-1995 (Pierce Chemical Co., Rockford, Ill.); Kuby, J., Immunology, 3.sup.rd Ed., W. H. Freeman & Co., New York, 1997.

Typically, a naturally occurring immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs.” The extent of the framework region and CDRs has been defined according to Kabat et al. (see, Kabat et al., Sequences of Proteins of Immunological Interest, U.S. Department of Health and Human Services, 1991) and the ImMunoGeneTics database (IMGT) (see, Lefranc, Nucleic Acids Res 29:207-9, 2001). The IMGT and Kabat databases are available online. The sequences of the framework regions of different light or heavy chains are relatively conserved within a species, such as humans. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are often identified by the chain in which the particular CDR is located. Thus, a V_{H} CDR3 (or H-CDR3) is located in the variable domain of the heavy chain of the antibody in which it is found, whereas a VL CDR1 (or L-CDR1) is the CDR1 from the variable domain of the light chain of the antibody in which it is found. An antibody that binds α-synuclein, for example, will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B-lymphocytes or by a cell into which the light and/or heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “chimeric antibody” contains structural elements from two or more different antibody molecules, often from different animal species. For example, a chimeric antibody can have framework residues from one species, such as human, and CDRs (which generally confer antigen binding) from another species, such as a murine antibody that specifically binds α-synuclein.

A “human” antibody (also called a “fully human” antibody) is an antibody that includes human framework regions and all of the CDRs from a human immunoglobulin. In one example, the framework and the CDRs are from the same originating human heavy and/or light chain amino acid sequence. However, frameworks from one human antibody can be engineered to include CDRs from a different human antibody. A “humanized” immunoglobulin is an immunoglobulin including a human framework region and one or more CDRs from a non-human (for example a mouse, rabbit, rat, or synthetic) immunoglobulin. The non-human immunoglobulin providing the CDRs is termed a “donor,” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by means of genetic engineering (see, e.g., U.S. Pat. No. 5,585,089).

A “single-domain antibody” (sdAb) or “nanobody” is an antibody fragment consisting of a single monomeric variable antibody domain. Like a whole antibody, it can bind selectively to a specific antigen. With a molecular weight of only 12-15 kDa, nanobodies are much smaller than common antibodies (150-160 kDa) which are composed of two heavy protein chains and two light chains, and even smaller than Fab fragments (^{˜}50 kDa, one light chain and half a heavy chain) and single-chain variable fragments (^{˜}25 kDa, two variable domains, one from a light and one from a heavy chain). The smaller size and single domain make nanobodies easier to transform into bacterial cells for bulk production, making them ideal for research purposes. A nanobody can be obtained by immunization of dromedaries, camels, llamas, alpacas or sharks with the desired antigen and subsequent isolation of the mRNA coding for heavy-chain antibodies. By reverse transcription and polymerase chain reaction, a gene library of nanobodies containing several million clones is produced. Screening techniques like phage display and ribosome display help to identify the clones binding the antigen.

A different method uses gene libraries from animals that have not been immunized beforehand. Such naïve libraries usually contain only antibodies with low affinity to the desired antigen, making it necessary to apply affinity maturation by random mutagenesis as an additional step.

When the most potent clones have been identified, their DNA sequence is optimized, for example to improve their stability towards enzymes. Another goal is humanization to prevent immunological reactions of the human organism against the antibody. Humanization is unproblematic because of the homology between camelid VHH and human VH fragments. The final step is the translation of the optimized nanobody in E. coli, S. cerevisiae or other suitable organisms.

Alternatively, nanobodies can be made from common murine or human IgG with four chains. The process is similar, comprising gene libraries from immunized or naïve donors and display techniques for identification of the most specific antigens. Monomerization is usually accomplished by replacing lipophilic by hydrophilic amino acids. The nanobodies can likewise be produced in E. coli, S. cerevisiae or other organisms.

An “intrabody” is an antibody that works within the cell to bind to an intracellular protein. Due to the lack of a reliable mechanism for bringing antibodies into a living cell from the extracellular environment, this typically requires the expression of the antibody within the target cell, which can be accomplished by gene therapy. As a result, intrabodies are defined as antibodies that have been modified for intracellular localization. For example, the antibody may remain in the cytoplasm, or it may have a nuclear localization signal, or it may undergo cotranslational translocation across the membrane into the lumen of the endoplasmic reticulum, provided that it is retained in that compartment through a KDEL sequence.

Because antibodies ordinarily are designed to be secreted from the cell, intrabodies often require special alterations, including the use of single-chain antibodies (scFvs), modification of immunoglobulin VL domains for hyperstability, selection of antibodies resistant to the more reducing intracellular environment, or expression as a fusion protein with maltose binding protein or other stable intracellular proteins. Such optimizations may improve the stability and structure of intrabodies.

Binding Affinity: Affinity of an antibody for an antigen. In one embodiment, affinity is calculated by a modification of the Scatchard method described by Frankel et al. (Mol. Immunol., 16:101-106, 1979). In another embodiment, binding affinity is measured by an antigen/antibody dissociation rate. In another embodiment, a high binding affinity is measured by a competition radioimmunoassay. In another embodiment, binding affinity is measured by ELISA. An antibody that “specifically binds” an antigen (such as α-synuclein) is an antibody that binds the antigen with high affinity and does not significantly bind other unrelated antigens.

Conservative variant: “Conservative” amino acid substitutions are those substitutions that do not substantially affect or decrease the affinity of a protein, such as an antibody to α-synuclein. For example, a monoclonal antibody that specifically binds α-synuclein can include at most about 1, at most about 2, at most about 5, at most about 10, or at most about 15 conservative substitutions and specifically bind a α-synuclein polypeptide. The term “conservative variant” also includes the use of a substituted amino acid in place of an unsubstituted parent amino acid, provided that antibody specifically binds α-synuclein. Non-conservative substitutions are those that reduce an activity or binding to α-synuclein.

Conservative amino acid substitution tables providing functionally similar amino acids are well known to one of ordinary skill in the art. The following six groups are examples of amino acids that are considered to be conservative substitutions for one another:

### 1) Alanine (A), Serine (S), Threonine (T);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V); and

### 6) Phenylalanine (F), Tyrosine (Y), Tryptophan (W).

Complementarity determining region (CDR): Amino acid sequences which together define the binding affinity and specificity of the natural Fv region of a native Ig binding site. The light and heavy chains of an Ig each have three CDRs, designated L-CDR1, L-CDR2, L-CDR3 and H-CDR1, H-CDR2, H-CDR3, respectively.

Degenerate Variant: In the context of the present disclosure, a “degenerate variant” refers to a polynucleotide encoding a α-synuclein polypeptide or an antibody that binds α-synuclein that includes a sequence that is degenerate as a result of the genetic code. There are 20 natural amino acids, most of which are specified by more than one codon. Therefore, all degenerate nucleotide sequences are included as long as the amino acid sequence of the α-synuclein polypeptide or antibody that binds α-synuclein encoded by the nucleotide sequence is unchanged.

Dementia with Lewy Bodies: also known as Lewy body dementia (LBD), diffuse Lewy body disease, cortical Lewy body disease, and senile dementia of Lewy type. A type of progressive neurodegenerative dementia closely associated with Parkinson's disease primarily affecting older adults. Its primary feature is a more rapid cognitive decline than with Parkinson's, which may lead to hallucinations, as well as varied attention and alertness when compared to a person's baseline function.

People with LBD display an inability to plan or a loss of analytical or abstract thinking and show markedly fluctuating cognition. Wakefulness varies from day to day, and alertness and short-term memory rise and fall. Persistent or recurring visual hallucinations with vivid and detailed imagery often are an early diagnostic symptom. The disorder is characterized anatomically by the presence of Lewy bodies, clumps of alpha-synuclein and ubiquitin protein in neurons, detectable in post mortem brain histology.

Diagnostic: Identifying the presence or nature of a pathologic condition, such as, but not limited to, Parkinson's disease. Diagnostic methods differ in their sensitivity and specificity. The “sensitivity” of a diagnostic assay is the percentage of diseased individuals who test positive (percent of true positives). The “specificity” of a diagnostic assay is one minus the false positive rate, where the false positive rate is defined as the proportion of those without the disease who test positive. While a particular diagnostic method may not provide a definitive diagnosis of a condition, it suffices if the method provides a positive indication that aids in diagnosis. “Prognostic” is the probability of development (e.g., severity) of a pathologic condition, such as cancer or metastasis.

Effector Molecule: The portion of a chimeric molecule that is intended to have a desired effect on a cell to which the chimeric molecule is targeted. Effector molecule is also known as an effector moiety (EM), therapeutic agent, or diagnostic agent, or similar terms.

Therapeutic agents include such compounds as nucleic acids, proteins, peptides, amino acids or derivatives, glycoproteins, radioisotopes, lipids, carbohydrates, or recombinant viruses. Nucleic acid therapeutic and diagnostic moieties include antisense nucleic acids, derivatized oligonucleotides for covalent cross-linking with single or duplex DNA, and triplex forming oligonucleotides. Alternatively, the molecule linked to a targeting moiety, such as an anti-α-synuclein antibody, may be an encapsulation system, such as a liposome or micelle that contains a therapeutic composition such as a drug, a nucleic acid (such as an antisense nucleic acid), or another therapeutic moiety that can be shielded from direct exposure to the circulatory system. Means of preparing liposomes attached to antibodies are well known to those of skill in the art (see, e.g., U.S. Pat. No. 4,957,735; and Connor et al., Pharm Ther 28:341-365, 1985). Diagnostic agents or moieties include radioisotopes and other detectable labels. Detectable labels useful for such purposes are also well known in the art, and include radioactive isotopes such as ³⁵S, ¹¹C, ¹³N, ¹⁵O, ¹⁸F, ¹⁹F, ⁹⁹mTc, ¹³¹I, ³H, ¹⁴C, ¹⁵N, ⁹⁰Y, ⁹⁹Tc, ¹¹¹In and ¹²⁵I, fluorophores, chemiluminescent agents, and enzymes.

Epitope: An antigenic determinant. These are particular chemical groups or peptide sequences on a molecule that are antigenic, i.e. that elicit a specific immune response. An antibody specifically binds a particular antigenic epitope on a polypeptide, such as α-synuclein.

Framework Region: Amino acid sequences interposed between CDRs. Framework regions include variable light and variable heavy framework regions. The framework regions serve to hold the CDRs in an appropriate orientation for antigen binding.

Host Cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Hybridoma: A hybrid cell for the production of monoclonal antibodies. A hybridoma is produced by fusion of an antibody-producing cell (such as a B cell obtained from an immunized animal, for example a mouse, rat or rabbit) and a myeloma cell.

Immune Response: A response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4⁺ response or a CD8⁺ response. In another embodiment, the response is a B cell response, and results in the production of specific antibodies.

Immunoconjugate: A covalent linkage of an effector molecule to an antibody or functional fragment thereof. The effector molecule can be, e.g., a detectable label. A “chimeric molecule” is a targeting moiety, such as a ligand or an antibody, conjugated (coupled) to an effector molecule. The term “conjugated” or “linked” refers to making two polypeptides into one contiguous polypeptide molecule. In one embodiment, an antibody is joined to an effector molecule. In another embodiment, an antibody joined to an effector molecule is further joined to a lipid or other molecule to a protein or peptide to increase its half-life in the body. The linkage can be either by chemical or recombinant means. In one embodiment, the linkage is chemical, wherein a reaction between the antibody moiety and the effector molecule has produced a covalent bond formed between the two molecules to form one molecule. A peptide linker (short peptide sequence) can optionally be included between the antibody and the effector molecule. Because immunoconjugates were originally prepared from two molecules with separate functionalities, such as an antibody and an effector molecule, they are also sometimes referred to as “chimeric molecules.” The term “chimeric molecule,” as used herein, therefore refers to a targeting moiety, such as a ligand or an antibody, conjugated (coupled) to an effector molecule.

Isolated: An “isolated” biological component, such as a nucleic acid, protein (including antibodies) or organelle, has been substantially separated or purified away from other biological components in the environment (such as a cell) in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Label: A detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a “labeled antibody” refers to incorporation of another molecule in the antibody. For example, the label is a detectable marker, such as the incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (as ³⁵S, ¹¹C, ¹³N, ¹⁵O, ¹⁸F, ¹⁹F, ⁹⁹mTc, ¹³¹I, ³H, ¹⁴C, ¹⁵N, ⁹⁰Y, ⁹⁹Tc, ¹¹¹In and ¹²⁵I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms of various lengths to reduce potential steric hindrance.

Linker: In some cases, a linker is a peptide within an antibody binding fragment (such as an Fv fragment) which serves to indirectly bond the variable heavy chain to the variable light chain. “Linker” can also refer to a peptide serving to link a targeting moiety, such as an antibody, to an effector molecule, such as a cytotoxin or a detectable label.

The terms “conjugating,” “joining,” “bonding” or “linking” refer to making two polypeptides into one contiguous polypeptide molecule, or to covalently attaching a radionuclide or other molecule to a polypeptide, such as an scFv. In the specific context, the terms include reference to joining a ligand, such as an antibody moiety, to an effector molecule. The linkage can be either by chemical or recombinant means. “Chemical means” refers to a reaction between the antibody moiety and the effector molecule such that there is a covalent bond formed between the two molecules to form one molecule.

Mammal: This term includes both human and non-human mammals. Similarly, the term “subject” includes both human and veterinary subjects.

Multiple system atrophy (MSA): a degenerative neurological disorder that depicts a group of disorders characterized by the neuronal degeneration mainly in the substantia nigra, striatum, autonomic nervous system and cerebellum. Many patients have symptoms and signs of cerebellar ataxia and parkinsonian manifestations. More than half of the patients with striatonigral degeneration have orthostatic hypotension, which proves at autopsy to be associated with loss of intermediolateral horn cells (origin of the presynaptic cholinergic sympathetic neurons) and of pigmented nuclei of the brainstem.

This combined parkinsonian and autonomic disorder is referred to as the Shy-Drager syndrome. In addition to orthostatic hypotension, other features of autonomic failure include impotence, loss of sweating, dry mouth and urinary retention and incontinence. Vocal cord palsy is an important and sometimes initial clinical manifestation of the disorder.

Both MRI and CT scanning frequently show atrophy of the cerebellum and pons in those with cerebellar features. The putamen is hypodense on T2-weighted MRI and may show an increased deposition of iron in Parkinsonian form. In cerebellar form, a “hot cross” sign has been emphasized; it reflects atrophy of the pontocereballar fibers that manifest in T2 signal intensity in atrophic pons.

MSA often presents with some of the same symptoms as Parkinson's disease. However, those with MSA generally show minimal if any response to the dopamine medications used for Parkinson's disease.

Multiple system atrophy can be explained as cell loss and gliosis or a proliferation of astrocytes in damaged areas of the central nervous system. This damage forms a scar which is then termed a glial scar. The presence of these inclusions (also known as Papp-Lantos bodies) in the movement, balance, and autonomic-control centers of the brain are the defining histopathologic hallmark of MSA. Recent studies have shown that the major filamentous component of glial and neuronal cytoplasmic inclusions is α-synuclein. Mutations in this substance may play a role in the disease. Tau proteins have been found in some GCIs.

Operably linked: A first nucleic acid sequence is operably linked with a second nucleic acid sequence when the first nucleic acid sequence is placed in a functional relationship with the second nucleic acid sequence. For instance, a promoter, such as the CMV promoter, is operably linked to a coding sequence if the promoter affects the transcription or expression of the coding sequence. Generally, operably linked DNA sequences are contiguous and, where necessary to join two protein-coding regions, in the same reading frame.

Parkinson's disease: is a long term disorder of the central nervous system that mainly affects the motor system. The symptoms generally come on slowly over time. Early in the disease, the most obvious are shaking, rigidity, slowness of movement, and difficulty with walking. Thinking and behavioral problems may also occur. Dementia becomes common in the advanced stages of the disease. Depression and anxiety are also common occurring in more than a third of people with PD. Other symptoms include sensory, sleep, and emotional problems. The main motor symptoms are collectively called “parkinsonism”, or a “parkinsonian syndrome.”

The cause of Parkinson's disease is believed to involve both genetic and environmental factors. Those with a family member affected are more likely to get the disease themselves. There is also an increased risk in people exposed to certain pesticides and among those who have had prior head injuries. The motor symptoms of the disease result from the death of cells in the substantia nigra, a region of the midbrain. This results in not enough dopamine in these areas. The reason for this cell death is involves the build-up of proteins into Lewy bodies in the neurons. Diagnosis of typical cases is mainly based on symptoms, with tests such as neuroimaging being used to rule out other diseases.

There is no cure for Parkinson's disease. Initial treatments is typically with the antiparkinson medication levodopa, with dopamine agonists being used once levodopa becomes less effective. As the disease progresses and neurons continue to be lost, these medications become less effective while at the same time they produce a complication marked by involuntary writhing movements.] Surgery to place the microelectrodes for deep brain stimulation has been used to reduce motor symptoms in severe cases where drugs are ineffective.

Pharmaceutical agent: A chemical compound or composition capable of inducing a desired therapeutic or prophylactic effect when properly administered to a subject or a cell.

Pharmaceutically acceptable carriers: The pharmaceutically acceptable carriers of use are conventional. Remington's Pharmaceutical Sciences, by E. W. Martin, Mack Publishing Co., Easton, Pa., 15th Edition, 1975, describes compositions and formulations suitable for pharmaceutical delivery of the antibodies disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Purified: The term purified does not require absolute purity; rather, it is intended as a relative term. Thus, for example, a purified peptide preparation is one in which the peptide or protein is more enriched than the peptide or protein is in its natural environment within a cell. In one embodiment, a preparation is purified such that the protein or peptide represents at least 50% of the total peptide or protein content of the preparation. Substantial purification denotes purification from other proteins or cellular components. A substantially purified protein is at least 60%, 70%, 80%, 90%, 95% or 98% pure. Thus, in one specific, non-limiting example, a substantially purified protein is 90% free of other proteins or cellular components.

Recombinant: A recombinant nucleic acid is one that has a sequence that is not naturally occurring or has a sequence that is made by an artificial combination of two otherwise separated segments of sequence. This artificial combination is often accomplished by chemical synthesis or by the artificial manipulation of isolated segments of nucleic acids, for example, by genetic engineering techniques.

Sample (or biological sample): A biological specimen containing genomic DNA, RNA (including mRNA), protein, or combinations thereof, obtained from a subject. Examples include, but are not limited to, peripheral blood, tissue, cells, urine, saliva, tissue biopsy, fine needle aspirate, surgical specimen, and autopsy material.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide or nucleic acid molecule will possess a relatively high degree of sequence identity when aligned using standard methods.

Methods of alignment of sequences for comparison are well known in the art. Various programs and alignment algorithms are described in: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237; Higgins and Sharp (1989) CABIOS 5:151; Corpet et al. (1988) Nucleic Acids Research 16:10881; and Pearson and Lipman (1988) Proc. Natl. Acad. Sci. U.S.A. 85:2444. Altschul et al. (1994) Nature Genet. 6:119, presents a detailed consideration of sequence alignment methods and homology calculations.

The NCBI Basic Local Alignment Search Tool (BLAST) (Altschul et al. (1990) J. Mol. Biol. 215:403) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of a V_{L} or a V_{H} of an antibody that specifically binds α-synuclein or a fragment thereof are typically characterized by possession of at least about 75%, for example at least about 80%, 90%, 95%, 96%, 97%, 98% or 99% sequence identity counted over the full length alignment with the amino acid sequence of the antibody using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Subject: Living multi-cellular vertebrate organisms, a category that includes both human and veterinary subjects, including human and non-human mammals.

Synthetic: Produced by artificial means in a laboratory, for example a monoclonal antibody produced by hybridoma technology or expressed from a cDNA construct.

Synucleinopathy: A neurodegenerative disease characterized by the abnormal accumulation of aggregates of α-synuclein proteins in neurons, nerve fibers, or glial cells. There are three main types of synucleinopathy: Parkinson's disease, dementia with Lewy bodies, and multiple system atrophy. Other rare disorders, such as various neuroaxonal dystrophies, also have α-synuclein pathologies.

Therapeutically effective amount: A quantity of a specific substance sufficient to achieve a desired effect in a subject being treated. For instance, this can be the amount necessary to inhibit or suppress growth of a tumor. In one embodiment, a therapeutically effective amount is the amount necessary to eliminate, reduce the size, or prevent metastasis of a tumor. When administered to a subject, a dosage will generally be used that will achieve target tissue concentrations (for example, in tumors) that has been shown to achieve a desired in vitro effect.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art.

### III. α-Synuclein-Specific Monoclonal Antibodies

Disclosed herein is the MJFR-14-6-4-2 antibody, a rabbit anti-α-synuclein filament antibody. This antibody is specific to a six amino acid C-terminal consensus motif on the α-synuclein amino acid sequence.

Complementary determining region (CDR) sequencing for the antibody identifies the specific amino acid residues of the antibody within the variable domain that directly/physically interact with the antigen. An antibody variable region has a heavy and light chain (designated V(H)/V(L)) containing CDRs and interface framework (FRM) amino acid residues which confer the strength and antigen binding affinity. An IgG serotype antibody has 2 variable regions, each with 3 potential CDRs for a potential total of 6 CDRs that collectively confer the specificity of the antibody's recognition of its antigen. Out of this, the following nomenclature is defined:

CDR1, CDR2, CDR3=complementarity determining region 1, 2, 3 etc. These are not necessarily sequentially designated in a linear sequence representation of the antibody protein (more on this below)

FRM1, 2, 3,=framework 1, 2, 3 regions (FRM1 associates with CDR1, FRM2 with CDR2, etc).

As antibodies mature to iteratively recognize their antigens with increasing affinity, CDRs are highly variable and their changes are what increase the specific, physical interaction with the antigen (the typified lock and key mechanism). FRMs are also located in the variable region but they are less malleable compared to CDRs. The FRMs don't change iteratively per se, but they impact the antibody:antigen interface by hinging/structurally shifting (determined by individual amino acid biochemical characteristics) when they encounter antigen to allow CDRs to maximally come into spatial proximity and thus physical contact with specific targets on the antigen.

Importantly, the CDR/FRM “pair” may not be proximal in linear amino acid sequence (so they don't align in the linear sequence data), but they are spatially proximal when the antibody protein is folded into its tertiary structure. This also why a given CDR/FRM pair don't necessarily “match” in terms of number of amino acid residues either. CDRs can vary from each other in their number of amino acids, as can FRMs, and the number of residues in a CDR/FRM pair often don't have the same number of amino acid residues.

ANTIBODY SEQUENCES
Amino acid sequence of heavy chain (SEQ ID NO: 1)
METGLRWLLLVAVLKGVQCQEQLVESGGDLVKPGASLTLTCTASGFSFSS
NYWMCWFRQAPGKGPEWIACIYAGNSGSTYYATWAKGRFTISKTSSTTVT
LQMTSLTAADTATYFCWRRGAYGYYGDLNLWGPGTLVTVSS
DNA sequence encoding heavy chain (SEQ ID NO: 2)
ATGGAGACTGGGCTGCGCTGGCTTCTCCTGGTCGCTGTGCTCAAAGGTGT
CCAGTGTCAGGAGCAGCTGGTGGAGTCCGGGGGAGACCTGGTCAAGCCTG
GGGCGTCCCTGACACTCACCTGCACAGCCTCTGGATTCTCCTTCAGTAGC
AACTACTGGATGTGCTGGTTCCGCCAGGCTCCAGGGAAGGGGCCGGAGTG
GATCGCATGCATTTATGCTGGTAATAGTGGTAGCACTTACTACGCGACCT
GGGCGAAAGGCCGATTCACCATCTCCAAAACCTCGTCGACCACGGTGACT
CTGCAAATGACCAGTCTGACAGCCGCGGACACGGCCACCTATTTCTGTTG
GAGAAGGGGTGCTTATGGATATTATGGTGATCTTAATTTGTGGGGCCCAG
GCACCCTGGTCACCGTCTCCTCAGGGCAACCTAAGGCTCCATCAGTCTTC
CCACTGGCCCCCTGCTGCGGGGACACACCCAGCTCCACGGTGACCCTGGG
CTGCCTGGTCAAAGGGTACCTCCCGGAGCCAGTGACCGTGACCTGGAACT
CGGGCACCCTCACCAATGGGGTACGCACCTTCCCGTCCGTCCGGCAGTCC
TCAGGCCTCTACTCGCTGAGCAGCGTGGTGAGCGTGACCTCAAGCAGCCA
GCCCGTCACCTGCAACGTGGCCCACCCAGCCACCAACACCAAAGTGGACA
AGACCGTTGCGCCCTCGACATGCAGCAAGCCCACGTGCCCACCCCCTGAA
CTCCTGGGGGGACCGTCTGTCTTCATCTTCCCCCCAAAACCCAAGGACAC
CCTCATGATCTCACGCACCCCCGAGGTCACATGCGTGGTGGTGGACGTGA
GCCAGGATGACCCCGAGGTGCAGTTCACATGGTACATAAACAACGAGCAG
NTGCGCACCGCCCGGGCCGCCGCTACGGGNGCAGCAGTTCAACAGCACGA
TCCGCGNNNGNCAGCNCCCTCCCCATCGCGCACNGNACTGGCTGAGGGCA
AGNAGTTCAAGTGCAAAGTCCANANNAGGCACTCCCGGCCCCATCNANAA
ANNNTCTNCAAANNNNANGGNNANNCCNNNNCNNNNCTANNNNGNNNTCC
GGNNGNNCNNANNNNCANGNNGNNANCNNNNNNCNNNNNNATNANGNNNN
NNNNCNNNNAANNNNNNNNNGNNNNNNN
Amino acid sequence of heavy chain FRM1
(SEQ ID NO: 3):
QEQLVESGGDLVKPGASLTLTCTASGFSFS
Amino acid sequence of heavy chain CDR1
(SEQ ID NO: 4):
SNYWMC
Amino acid sequence of heavy chain FRM2
(SEQ ID NO: 5):
WFRQAPGKGPEWIA
Amino acid sequence of heavy chain CDR2
(SEQ ID NO: 6):
CIYAGNSGSTYYATWAKG
Amino acid sequence of heavy chain FRM3
(SEQ ID NO: 7):
RFTISKTSSTTVTLQMTSLTAADTATYFCWR
Amino acid sequence of heavy chain CDR3
(SEQ ID NO: 8):
RGAYGYYGDLNL
Amino acid sequence of heavy chain FRM4
(SEQ ID NO: 9)
WGPGTLVTVSS
Amino acid sequence of light chain
(SEQ ID NO: 10):
MDTRAPTQLLGLLLLWLPGATFAQVLTQTASSVSAAVGGTVTISCQSSQS
VYKNNYLAWYQQKPGQPPNLLIYDASTLASGVSSRFRGSGSGTQFTLTIS
GVQCDDAATYYCQGGFPCRTADCNVFGGGTEVVVK
DNA sequence encoding light chain (SEQ ID NO: 11)
ATGGACACGAGGGCCCCCACTCAGCTGCTGGGGCTCCTGCTGCTCTGGCT
CCCAGGTGCCACATTTGCCCAAGTGCTGACCCAGACTGCATCGTCCGTGT
CTGCAGCTGTGGGAGGCACAGTCACCATCAGTTGCCAGTCCAGTCAGAGT
GTTTATAAGAACAACTACTTAGCCTGGTATCAGCAGAAACCAGGGCAGCC
TCCCAACCTCCTGATCTATGATGCATCCACTCTGGCATCTGGGGTCTCAT
CGCGGTTCAGAGGCAGTGGATCTGGGACACAGTTCACTCTCACCATCAGC
GGCGTGCAGTGTGACGATGCTGCCACTTACTACTGTCAAGGCGGATTTCC
TTGTCGTACTGCTGATTGTAATGTTTTCGGCGGAGGGACCGAGGTGGTGG
TCAAAGGTGATCCAGTTGCACCTACTGTCCTCATCTTCCCACCAGCTGCT
GATCAGGTGGCAACTGGAACAGTCACCATCGTGTGTGTGGCGAATAAATA
CTTTCCCGATGTCACCGTCACCTGGGAGGTGGATGGCACCACCCAAACAA
CTGGCATCGAGAACAGTAAAACACCGCAGAATTCTGCAGATTGTACCTAC
AACCTCAGCAGCACTCTGACACTGACCAGCACACAGTACAACAGCCACAA
AGAGTACACCTGCAAGGTGACCCAGGGCACGACCTCAGTCGTCCAGAGCT
TCAATAGGGGTGACTGTTAGAGCGAGAGCGGCCGCTCGAGGCCGGCAAGG
CCGGATCCCCCGACCTCGACCTCTGGCTAATAAAGGAAATTTATTTTCAT
TGCAATAGTGTGTTGGAATTTTTTGTGTCTCTCACTCGGAANGGACATAT
GGGANGGCAAATCATTTGGTCGAGATCCCTCGGANATCTCTAGCTAGAGG
ATCGATCCCCGCCCCGGANGAACTAANNNTGACTACGACATCTCTGCCCC
TNCNTCNCGGGGCANNGCATGTAATCCCT
Amino acid sequence of light chain FRM1
(SEQ ID NO: 12)
AQVLTQTASSVSAAVGGTVTISC
Amino acid sequence of light chain CDR1
(SEQ ID NO: 13):
QSSQSVYKNNYLA
Amino acid sequence of light chain FRM2
(SEQ ID NO: 14):
WYQQKPGQPPNLLIY
Amino acid sequence of light chain CDR2
(SEQ ID NO: 15):
DASTLAS
Amino acid sequence of light chain FRM3
(SEQ ID NO: 16):
GVSSRFRGSGSGTQFTLTISGVQCDDAATYYC
Amino acid sequence of light chain CDR3
(SEQ ID NO: 17):
QGGFPCRTADCNV
Amino acid sequence of light chain FRM4
(SEQ ID NO: 18):
FGGGTEVVVK

In some embodiments, the monoclonal antibody that binds, such as specifically binds, α-synuclein is a single domain antibody.

In some embodiments, the monoclonal antibody that binds, such as specifically binds, α-synuclein is a Fab fragment, a Fab′ fragment, a F(ab)′₂ fragment, a single chain variable fragment (scFv), or a disulfide stabilized variable fragment (dsFv). In other embodiments, the antibody is an immunoglobulin molecule. In particular examples, the antibody is an IgG.

In some embodiments, the monoclonal antibody is chimeric or synthetic.

In some embodiments, the disclosed antibodies bind α-synuclein (soluble or cell-surface α-synuclein) with a dissociation constant (K_{d}) in the high pm (^{˜}50-100) to low nm range. In one embodiment, the monoclonal antibodies bind α-synuclein with a binding affinity of about 30 pM.

The monoclonal antibodies disclosed herein can be labeled, such as with a fluorescent, enzymatic, or radioactive label.

Also provided are fusion proteins comprising an antibody disclosed herein and a heterologous protein. In some examples, the heterologous protein is an Fc protein. In one non-limiting example, the Fc protein is a human Fc protein, such as human IgGγ1 Fc.

Further provided herein are compositions comprising a therapeutically effective amount of a disclosed antibody, immunoconjugate or fusion protein and a pharmaceutically acceptable carrier.

Also provided herein are isolated nucleic acid molecules encoding the disclosed monoclonal antibodies, immunoconjugates and fusion proteins. In some examples, the isolated nucleic acid molecule is operably linked to a promoter.

Also provided are expression vectors comprising the isolated nucleic acid molecules disclosed herein. Isolated host cells comprising the nucleic acid molecules or vectors are also provided herein.

### V. Antibodies and Antibody Fragments

The monoclonal antibodies disclosed herein can be of any isotype. The monoclonal antibody can be, for example, an IgM or an IgG antibody, such as IgG₁ or an IgG₂. The class of an antibody that specifically binds α-synuclein can be switched with another (for example, IgG can be switched to IgM), according to well-known procedures. Class switching can also be used to convert one IgG subclass to another, such as from IgG₁ to IgG₂.

Antibody fragments are also encompassed by the present disclosure, such as single-domain antibodies (e.g., VH domain antibodies), Fab, F(ab′)₂, and Fv. These antibody fragments retain the ability to selectively bind with the antigen. These fragments include:

(1) Fab, the fragment which contains a monovalent antigen-binding fragment of an antibody molecule, can be produced by digestion of whole antibody with the enzyme papain to yield an intact light chain and a portion of one heavy chain;
(2) Fab′, the fragment of an antibody molecule can be obtained by treating whole antibody with pepsin, followed by reduction, to yield an intact light chain and a portion of the heavy chain; two Fab′ fragments are obtained per antibody molecule;
(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;
(4) Fv, a genetically engineered fragment containing the variable region of the light chain and the variable region of the heavy chain expressed as two chains;
(5) Single chain antibody (such as scFv), a genetically engineered molecule containing the variable region of the light chain, the variable region of the heavy chain, linked by a suitable polypeptide linker as a genetically fused single chain molecule;
(6) A dimer of a single chain antibody (scFV₂), defined as a dimer of a scFV (also known as a “miniantibody”); and
(7) VH single-domain antibody, an antibody fragment consisting of a heavy chain variable domain.

Methods of making these fragments are known in the art (see for example, Harlow and Lane, Antibodies: A Laboratory Manual, Cold Spring Harbor Laboratory, New York, 1988).

In some cases, antibody fragments can be prepared by proteolytic hydrolysis of the antibody or by expression in a host cell (such as E. coli) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. Nos. 4,036,945 and 4,331,647).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

One of skill will realize that conservative variants of the antibodies can be produced. Such conservative variants employed in antibody fragments, such as dsFv fragments or in scFv fragments, will retain critical amino acid residues necessary for correct folding and stabilizing between the V_{H} and the V_{L} regions, and will retain the charge characteristics of the residues in order to preserve the low pl and low toxicity of the molecules Amino acid substitutions (such as at most one, at most two, at most three, at most four, or at most five amino acid substitutions) can be made in the V_{H} and/or the V_{L} regions to increase yield. Conservative amino acid substitution tables providing functionally similar amino acids are well known to one of ordinary skill in the art. The following six groups are examples of amino acids that are considered to be conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

### VI. Immunoconjugates and Fusion Proteins

The disclosed monoclonal antibodies specific for α-synuclein can be conjugated to a therapeutic agent or effector molecule Immunoconjugates include, but are not limited to, molecules in which there is a covalent linkage of a therapeutic agent to an antibody. A therapeutic agent is an agent with a particular biological activity directed against a particular target molecule or a cell bearing a target molecule. One of skill in the art will appreciate that therapeutic agents can include various drugs, encapsulating agents (such as liposomes) which themselves contain pharmacological compositions, radioactive agents such as ¹²⁵I, ³²P, ¹⁴C, ³H and ³⁵S and other labels, target moieties and ligands. The choice of a particular therapeutic agent depends on the particular target molecule or cell, and the desired biological effect.

With the therapeutic agents and antibodies described herein, one of skill can readily construct a variety of clones containing functionally equivalent nucleic acids, such as nucleic acids which differ in sequence but which encode the same effector moiety or antibody sequence. Thus, the present disclosure provides nucleic acids encoding antibodies and conjugates and fusion proteins thereof.

Effector molecules can be linked to an antibody of interest using any number of means known to those of skill in the art. Both covalent and noncovalent attachment means may be used. The procedure for attaching an effector molecule to an antibody varies according to the chemical structure of the effector. Polypeptides typically contain a variety of functional groups; such as carboxylic acid (—COOH), free amine (—NH₂) or sulfhydryl (—SH) groups, which are available for reaction with a suitable functional group on an antibody to result in the binding of the effector molecule. Alternatively, the antibody is derivatized to expose or attach additional reactive functional groups. The derivatization may involve attachment of any of a number of known linker molecules. The linker can be any molecule used to join the antibody to the effector molecule. The linker is capable of forming covalent bonds to both the antibody and to the effector molecule. Suitable linkers are well known to those of skill in the art and include, but are not limited to, straight or branched-chain carbon linkers, heterocyclic carbon linkers, or peptide linkers. Where the antibody and the effector molecule are polypeptides, the linkers may be joined to the constituent amino acids through their side groups (such as through a disulfide linkage to cysteine) or to the alpha carbon amino and carboxyl groups of the terminal amino acids.

In some circumstances, it is desirable to free the effector molecule from the antibody when the immunoconjugate has reached its target site. Therefore, in these circumstances, immunoconjugates will comprise linkages that are cleavable in the vicinity of the target site. Cleavage of the linker to release the effector molecule from the antibody may be prompted by enzymatic activity or conditions to which the immunoconjugate is subjected either inside the target cell or in the vicinity of the target site.

In view of the large number of methods that have been reported for attaching a variety of radiodiagnostic compounds, radiotherapeutic compounds, label (such as enzymes or fluorescent molecules) drugs, toxins, and other agents to antibodies one skilled in the art will be able to determine a suitable method for attaching a given agent to an antibody or other polypeptide.

The antibodies or antibody fragments disclosed herein can be derivatized or linked to another molecule (such as another peptide or protein). In some cases, the antibody or antibody fragment (such as a VH domain) is fused to a heterologous protein, for example an Fc protein.

In general, the antibodies or portion thereof is derivatized such that the binding to the target antigen is not affected adversely by the derivatization or labeling. For example, the antibody can be functionally linked (by chemical coupling, genetic fusion, noncovalent association or otherwise) to one or more other molecular entities, such as another antibody (for example, a bispecific antibody or a diabody), a detection agent, a pharmaceutical agent, and/or a protein or peptide that can mediate association of the antibody or antibody portion with another molecule (such as a streptavidin core region or a polyhistidine tag).

One type of derivatized antibody is produced by cross-linking two or more antibodies (of the same type or of different types, such as to create bispecific antibodies). Suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate). Such linkers are commercially available.

An antibody that binds (for example specifically binds) α-synuclein or a fragment thereof can be labeled with a detectable moiety. Useful detection agents include fluorescent compounds, including fluorescein, fluorescein isothiocyanate, rhodamine, 5-dimethylamine-1-naphthalenesulfonyl chloride, phycoerythrin, lanthanide phosphors and the like. Bioluminescent markers are also of use, such as luciferase, Green fluorescent protein (GFP), Yellow fluorescent protein (YFP). An antibody can also be labeled with enzymes that are useful for detection, such as horseradish peroxidase, β-galactosidase, luciferase, alkaline phosphatase, glucose oxidase and the like. When an antibody is labeled with a detectable enzyme, it can be detected by adding additional reagents that the enzyme uses to produce a reaction product that can be discerned. For example, when the agent horseradish peroxidase is present the addition of hydrogen peroxide and diaminobenzidine leads to a colored reaction product, which is visually detectable. An antibody may also be labeled with biotin, and detected through indirect measurement of avidin or streptavidin binding. It should be noted that the avidin itself can be labeled with an enzyme or a fluorescent label.

An antibody may be labeled with a magnetic agent, such as gadolinium. Antibodies can also be labeled with lanthanides (such as europium and dysprosium), and manganese. Paramagnetic particles such as superparamagnetic iron oxide are also of use as labels. An antibody may also be labeled with a predetermined polypeptide epitopes recognized by a secondary reporter (such as leucine zipper pair sequences, binding sites for secondary antibodies, metal binding domains, epitope tags). In some embodiments, labels are attached by spacer arms of various lengths to reduce potential steric hindrance.

An antibody can also be labeled with a radiolabeled amino acid. The radiolabel may be used for both diagnostic and therapeutic purposes. For instance, the radiolabel may be used to detect α-synuclein by x-ray, emission spectra, or other diagnostic techniques. Examples of labels for polypeptides include, but are not limited to, the following radioisotopes or radionucleotides: ³H, ¹⁴C, ¹⁵N, ³⁵S, ⁹⁰Y, ⁹⁹Tc, ¹¹¹In, ¹²⁵I, ¹³¹I.

An antibody can also be derivatized with a chemical group such as polyethylene glycol (PEG), a methyl or ethyl group, or a carbohydrate group. These groups may be useful to improve the biological characteristics of the antibody, such as to increase serum half-life or to increase tissue binding.

The antibodies described herein can also be used to target any number of different diagnostic or therapeutic compounds to cells expressing α-synuclein on their surface. Thus, an antibody of the present disclosure can be attached directly or via a linker to a drug that is to be delivered directly to cells expressing cell-surface α-synuclein. This can be done for therapeutic, diagnostic or research purposes. Therapeutic agents include such compounds as nucleic acids, proteins, peptides, amino acids or derivatives, glycoproteins, radioisotopes, lipids, carbohydrates, or recombinant viruses. Nucleic acid therapeutic and diagnostic moieties include antisense nucleic acids, derivatized oligonucleotides for covalent cross-linking with single or duplex DNA, and triplex forming oligonucleotides.

Alternatively, the molecule linked to an anti-α-synuclein antibody can be an encapsulation system, such as a liposome or micelle that contains a therapeutic composition such as a drug, a nucleic acid (for example, an antisense nucleic acid), or another therapeutic moiety that is preferably shielded from direct exposure to the circulatory system. Means of preparing liposomes attached to antibodies are well known to those of skill in the art (see, for example, U.S. Pat. No. 4,957,735; Connor et al., Pharm. Ther. 28:341-365, 1985).

Antibodies described herein can also be covalently or non-covalently linked to a detectable label. Detectable labels suitable for such use include any composition detectable by spectroscopic, photochemical, biochemical, immunochemical, electrical, optical or chemical means. Useful labels include magnetic beads, fluorescent dyes (for example, fluorescein isothiocyanate, Texas red, rhodamine, green fluorescent protein, and the like), radiolabels (for example, ³H, ¹²⁵I, ³⁵S, ¹⁴C, or ³²P), enzymes (such as horseradish peroxidase, alkaline phosphatase and others commonly used in an ELISA), and colorimetric labels such as colloidal gold or colored glass or plastic (such as polystyrene, polypropylene, latex, and the like) beads.

Means of detecting such labels are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

### VII. Compositions and Methods of Use

Compositions are provided that include one or more of the disclosed antibodies that bind (for example specifically bind) α-synuclein in a carrier. Compositions comprising fusion proteins, immunoconjugates or immunotoxins are also provided. The compositions can be prepared in unit dosage forms for administration to a subject. The amount and timing of administration are at the discretion of the treating clinician to achieve the desired outcome. The antibody can be formulated for systemic or local (such as intracerebral) administration. In one example, the antibody is formulated for parenteral administration, such as intravenous administration.

The compositions for administration can include a solution of the antibody dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs.

A typical pharmaceutical composition for intravenous administration includes about 0.1 to 10 mg of antibody per subject per day. Dosages from 0.1 up to about 100 mg per subject per day may be used, particularly if the agent is administered to a secluded site and not into the circulatory or lymph system, such as into a body cavity or into a lumen of an organ. Actual methods for preparing administrable compositions will be known or apparent to those skilled in the art and are described in more detail in such publications as Remington's Pharmaceutical Science, 19th ed., Mack Publishing Company, Easton, Pa. (1995).

Antibodies may be provided in lyophilized form and rehydrated with sterile water before administration, although they are also provided in sterile solutions of known concentration. The antibody solution is then added to an infusion bag containing 0.9% sodium chloride, USP, and in some cases administered at a dosage of from 0.5 to 15 mg/kg of body weight. Considerable experience is available in the art in the administration of antibody drugs, which have been marketed in the U.S. since the approval of RITUXAN® in 1997. Antibodies can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30-minute period if the previous dose was well tolerated.

Controlled release parenteral formulations can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, Pa., (1995). Particulate systems include microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, J., Colloidal Drug Delivery Systems, J. Kreuter, ed., Marcel Dekker, Inc., New York, N.Y., pp. 219-342 (1994); and Tice & Tabibi, Treatise on Controlled Drug Delivery, A. Kydonieus, ed., Marcel Dekker, Inc. New York, N.Y., pp. 315-339, (1992).

Polymers can be used for ion-controlled release of the antibody compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa. (1993)). Numerous additional systems for controlled delivery of therapeutic proteins are known (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

### A. Therapeutic Methods

The antibodies, compositions, fusion proteins and immunoconjugates disclosed herein can be administered to slow or inhibit the growth of Lewy bodies or inhibit the aggregation of α-synuclein. In these applications, a therapeutically effective amount of an antibody is administered to a subject in an amount sufficient to inhibit aggregation of α-synuclein or growth of Lewy bodies. Suitable subjects may include those diagnosed with a synucleinopathy such as Parkinson's disease, dementia with Lewy bodies, or multiple system atrophy.

In one non-limiting embodiment, provided herein is a method of treating a subject with a synucleinopathy by selecting a subject having a synucleinopathy and administering to the subject a therapeutically effective amount of an antibody, composition, fusion protein or immunoconjugate disclosed herein.

Also provided herein is a method of inhibiting the aggregation of α-synuclein by selecting a subject having a synucleinopathy and administering to the subject a therapeutically effective amount of an antibody, composition, fusion protein or immunoconjugate disclosed herein.

A therapeutically effective amount of a α-synuclein-specific antibody, fusion protein, composition or immunoconjugate will depend upon the severity of the disease and the general state of the patient's health. A therapeutically effective amount of the antibody is that which provides either subjective relief of a symptom(s) or an objectively identifiable improvement as noted by the clinician or other qualified observer.

### B. Methods for Diagnosis and Detection

Methods are provided herein for detecting expression of α-synuclein in vitro or in vivo. In some cases, α-synuclein expression is detected in a biological sample. The sample can be any sample, including, but not limited to, tissue from biopsies, autopsies and pathology specimens. Biological samples also include sections of tissues, for example, frozen sections taken for histological purposes. Biological samples further include body fluids, such as blood, serum, plasma, sputum, spinal fluid or urine. A biological sample is typically obtained from a mammal, such as a human or non-human primate.

In one embodiment, provided is a method of determining if a subject has a synucleinopathy by contacting a sample from the subject with a monoclonal antibody disclosed herein; and detecting binding of the antibody to the sample. An increase in binding of the antibody to the sample as compared to binding of the antibody to a control sample identifies the subject as having cancer.

In another embodiment, provided is a method of confirming a diagnosis of a synucleinopathy in a subject by contacting a sample from a subject diagnosed with a synucleinopathy with a monoclonal antibody disclosed herein; and detecting binding of the antibody to the sample. An increase in binding of the antibody to the sample as compared to binding of the antibody to a control sample confirms the diagnosis of a synucleinopathy in the subject.

In some examples of the disclosed methods, the monoclonal antibody is directly labeled. In some examples, the methods further include contacting a second antibody that specifically binds the monoclonal antibody with the sample; and detecting the binding of the second antibody. An increase in binding of the second antibody to the sample as compared to binding of the second antibody to a control sample detects cancer in the subject or confirms the diagnosis of a synucleinopathy in the subject.

In some cases, the synucleinopathy is Parkinson's disease, dementia with Lewy bodies, multiple system atrophy, or any other type of synucleinopathy that expresses α-synuclein.

In some examples, the control sample is a sample from a subject without a synucleinopathy. In particular examples, the sample is a blood or tissue sample.

In some cases, the antibody that binds (for example specifically binds) α-synuclein is directly labeled with a detectable label. In another embodiment, the antibody that binds (for example, specifically binds) α-synuclein (the first antibody) is unlabeled and a second antibody or other molecule that can bind the antibody that specifically binds α-synuclein is labeled. As is well known to one of skill in the art, a second antibody is chosen that is able to specifically bind the specific species and class of the first antibody. For example, if the first antibody is a human IgG, then the secondary antibody may be an anti-human-IgG. Other molecules that can bind to antibodies include, without limitation, Protein A and Protein G, both of which are available commercially.

Suitable labels for the antibody or secondary antibody are described above, and include various enzymes, prosthetic groups, fluorescent materials, luminescent materials, magnetic agents and radioactive materials. Non-limiting examples of suitable enzymes include horseradish peroxidase, alkaline phosphatase, beta-galactosidase, or acetylcholinesterase. Non-limiting examples of suitable prosthetic group complexes include streptavidin/biotin and avidin/biotin. Non-limiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary a magnetic agent is gadolinium, and non-limiting exemplary radioactive labels include ¹²⁵I, ¹³¹I, ³⁵S or ³H.

In an alternative embodiment, α-synuclein can be assayed in a biological sample by a competition immunoassay utilizing α-synuclein standards labeled with a detectable substance and an unlabeled antibody that specifically binds α-synuclein. In this assay, the biological sample, the labeled α-synuclein standards and the antibody that specifically bind α-synuclein are combined and the amount of labeled α-synuclein standard bound to the unlabeled antibody is determined. The amount of α-synuclein in the biological sample is inversely proportional to the amount of labeled α-synuclein standard bound to the antibody that specifically binds α-synuclein.

The immunoassays and method disclosed herein can be used for a number of purposes. In one embodiment, the antibody that specifically binds α-synuclein may be used to detect the production of α-synuclein in cells in cell culture. In another embodiment, the antibody can be used to detect the amount of α-synuclein in a biological sample, such as a tissue sample, or a blood or serum sample. In some examples, the α-synuclein is soluble α-synuclein (e.g. α-synuclein in a cell culture supernatant or soluble α-synuclein in a body fluid sample, such as a blood or serum sample).

In one embodiment, a kit is provided for detecting α-synuclein in a biological sample, such as a blood sample or tissue sample. For example, to confirm a diagnosis in a subject, a biopsy can be performed to obtain a tissue sample for histological examination. Alternatively, a blood sample can be obtained to detect the presence of soluble α-synuclein protein or fragment. Kits for detecting a polypeptide will typically comprise a monoclonal antibody that specifically binds α-synuclein, such as any of the antibodies disclosed herein. In some embodiments, an antibody fragment, such as an scFv fragment, a VH domain, or a Fab is included in the kit. In a further embodiment, the antibody is labeled (for example, with a fluorescent, radioactive, or an enzymatic label).

In one embodiment, a kit includes instructional materials disclosing means of use of an antibody that binds α-synuclein. The instructional materials may be written, in an electronic form (such as a computer diskette or compact disk) or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

In one embodiment, the diagnostic kit comprises an immunoassay. Although the details of the immunoassays may vary with the particular format employed, the method of detecting α-synuclein in a biological sample generally includes the steps of contacting the biological sample with an antibody which specifically reacts, under immunologically reactive conditions, to a α-synuclein polypeptide. The antibody is allowed to specifically bind under immunologically reactive conditions to form an immune complex, and the presence of the immune complex (bound antibody) is detected directly or indirectly.

Methods of determining the presence or absence of an antigen are well known in the art. For example, the antibodies can be conjugated to other compounds including, but not limited to, enzymes, magnetic beads, colloidal magnetic beads, haptens, fluorochromes, metal compounds, radioactive compounds or drugs. The antibodies can also be utilized in immunoassays such as but not limited to radioimmunoassays (RIAs), ELISA, or immunohistochemical assays. The antibodies can also be used for fluorescence activated cell sorting (FACS). FACS employs a plurality of color channels, low angle and obtuse light-scattering detection channels, and impedance channels, among other more sophisticated levels of detection, to separate or sort cells (see U.S. Pat. No. 5,061,620). Any of the monoclonal antibodies that bind α-synuclein, as disclosed herein, can be used in these assays. Thus, the antibodies can be used in a conventional immunoassay, including, without limitation, an ELISA, an RIA, FACS, tissue immunohistochemistry, Western blot or immunoprecipitation.

The following examples are provided to illustrate certain particular features and/or embodiments. These examples should not be construed to limit the disclosure to the particular features or embodiments described.

### EXAMPLES

### Example 1: PEPperMAP Linear and Conformational Epitope Mappings of Rabbit Monoclonal Antibody MJFR 14-6-4-2 Against Alpha-Synuclein

Microarray Content: The protein sequence of α-synuclein was elongated by neutral GSGSGSG linkers at the C- and N-terminus to avoid truncated peptides. The elongated sequence was translated into 15 amino acid linear peptides with a peptide-peptide overlap of 14 amino acids. The resulting linear peptide microarrays contained 140 different peptides printed in duplicate (280 peptides spots) and were framed by additional HA (YPYDVPDYAG) control peptides (74 spots).

For the conformational epitope mappings, the elongated sequence was translated into 7, 10, and 13 amino acid peptides with peptide-peptide overlaps of 6, 9 and 12 amino acids. After peptide synthesis, all peptides were cyclized via a thioether linkage between a C-terminal cysteine side chain thiol group and an appropriately modified N-terminus. The resulting conformational peptide microarrays contained 435 different peptides printed in duplicate (870 peptides spots) and were framed by additional HA (YPYDVPDYAG) control peptides (86 spots).

Samples: Rabbit anti-α-synuclein filament antibody MJFR-14-6-4-2.

Washing Buffer: PBS, pH 7.4 with 0.05% Tween 20 (3×1 min after each assay) for the linear epitope mapping, PBS, pH 7.4 with 0.005% Tween 20 (2×10 sec after each assay) for the conformational epitope mapping.
Blocking Buffer: Rockland blocking buffer MB-070 in washing buffer (30 min before the first assay).
Incubation Buffer: Washing buffer with 10% blocking buffer.
Assay Conditions: Antibody concentrations of 1 μg/ml, 10 μg/ml and 100 μg/ml (MJF-R13 (8-8)) or dilutions of 1:1000 and 1:100 (MJFR-14-6-4-2) in incubation buffer; incubation for 16 h at 4° C. and shaking at 140 rpm.
Secondary Antibody: Sheep anti-rabbit IgG (H+L) DyLight680 (1:5000); 45 min staining in incubation buffer at RT
Control Antibody: Mouse monoclonal anti-HA (12CA5) DyLight800 (1:2000); 45 min staining in incubation buffer at RT.
Scanner: LI-COR Odyssey Imaging System; scanning offset 0.65 mm, resolution 21 μm, scanning intensities of 7/7 (red=700 nm/green=800 nm).

### Results:

Incubation with rabbit monoclonal antibody MJFR-14-6-4-2 at dilutions of 1:1000 and 1:100 was followed by staining with secondary and control antibodies and read out at scanning intensities of 7/7 (red/green). We observed a clear monoclonal response of rabbit monoclonal antibody MJFR-14-6-4-2 with the linear α-synuclein peptides with the C-terminal consensus motif YQDYEP with high signal-to-noise ratios.

The epitope of the MJF-R14 aSyn filament conformation-specific antibody mapped to a hexapeptide—YQDYEP—corresponding to amino acid positions 133-138 in the C-terminal (FIG. 2, 3).

We observed a clear monoclonal response of rabbit monoclonal antibody MJFR-14-6-4-2 with the cyclic constrained α-synuclein peptides with the C-terminal consensus motif DYEP, YQDYEP and EEGYQDYEP (FIG. 4) with high signal-to-noise ratios; the signal against peptide MPVDPDNEAYE also exhibited a heterogeneous spot morphology and was based on a microarray artifact

### DISCUSSION AND CONCLUSION

The PEPperMAP® Linear Epitope Mappings of rabbit anti-α-synuclein filament antibody MJFR-14-6-4-2 were performed with 15 aa peptides of α-synuclein with a peptide-peptide overlap of 14 aa; the corresponding PEPperMAP® Conformational Epitope Mappings were performed with 7, 10 and 13 amino acid cyclic constrained α-synuclein peptides with peptide-peptide overlaps of 6, 9 and 12 amino acids. The α-synuclein peptide microarray variants were incubated with the antibody samples at dilutions of 1:1000 and 1:100 (MJFR-14-6-4-2) in incubation buffer followed by staining with the secondary sheep anti-rabbit IgG (H+L) DyLight680 antibody and read-out with a LI-COR Odyssey Imaging System. Quantification of spot intensities and peptide annotation were done with PepSlide® Analyzer.

Pre-staining of both α-synuclein peptide microarray variants with secondary and control antibodies did not reveal any background interaction of the antigen-derived peptides that could interfere with the main assays. In contrast incubation with the antibody samples resulted in the following observations:

Rabbit monoclonal antibody MJFR-14-6-4-2 showed clear and strong monoclonal response against peptides with the C-terminal consensus motif YQDYEP with both the linear and the cyclic constrained α-synuclein; a clear conformational contribution was not observed; however, a strong decrease of spot intensities from linear peptide AYEMPSEEGYQDYEP to YEMPSEEGYQDYEPE may hint at an induced conformation by the C-terminal proline that was significantly disturbed by a shift of the proline to the N-terminus of the peptide, and hence possibly explain the observed dot blot activity.

### Example 2: α-Synuclein-Specific Monoclonal Antibodies for Detecting Synucleinopathy in a Subject or Confirming the Diagnosis of Synucleinopathy in a Subject

This example describes the use of α-synuclein-specific monoclonal antibodies, such as the monoclonal antibodies disclosed herein for the detection of a synucleinopathy in a subject. This example further describes the use of these antibodies to confirm the diagnosis of a synucleinopathy in a subject.

A blood sample is obtained from the patient diagnosed with, or suspected of having a synucleinopathy (such as Parkinson's disease, dementia with Lewy bodies, or multiple system atrophy). A blood sample taken from a patient that does not have a synucleinopathy can be used as a control. An ELISA is performed to detect the presence of soluble α-synuclein in the blood sample. Proteins present in the blood samples (the patient sample and control sample) are immobilized on a solid support, such as a 96-well plate, according to methods well known in the art (see, for example, Robinson et al., Lancet 362:1612-1616, 2003). Following immobilization, α-synuclein-specific monoclonal antibody directly labeled with a fluorescent marker is applied to the protein-immobilized plate. The plate is washed in an appropriate buffer, such as PBS, to remove any unbound antibody and to minimize non-specific binding of antibody. Fluorescence can be detected using a fluorometric plate reader according to standard methods. An increase in fluorescence intensity of the patient sample, relative to the control sample, indicates the anti-α-synuclein antibody specifically bound proteins from the blood sample, thus detecting the presence of α-synuclein protein in the sample. Detection of α-synuclein protein in the patient sample indicates the patient has a synucleinopathy, or confirms diagnosis of a synucleinopathy in the subject.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

What is claimed is:

1. An isolated rabbit monoclonal antibody that specifically binds the C-terminal consensus motif YQDYEP of human α-synuclein

2. The antibody of claim 1, wherein the amino acid sequence of the antibody is at least 90% or at least 95% identical to SEQ ID NO: 1 or SEQ ID NO: 10.

3. The antibody of claim 1, wherein the amino acid sequence of the antibody comprises SEQ ID NO: 2 or SEQ ID NO: 10.

4. The antibody of claim 1, wherein the antibody is chimeric or synthetic.

5. The antibody of claim 1, wherein the antibody is a nanobody.

6. The antibody of claim 1, wherein the antibody is labeled.

7. The antibody of claim 6, wherein the label is a fluorescent, enzymatic, or radioactive label.

8. An isolated immunoconjugate comprising antibody of claim 1 and an effector molecule.

9. A fusion protein comprising the antibody of claim 1 and a heterologous protein.

10. The fusion protein of claim 9, wherein the heterologous protein is a human Fc protein.

11. A composition comprising an antibody of claim 1 and a carrier therefor.

12. A method comprising administering to a subject an antibody according to claim 1.

13. A method of detecting α-synuclein in a biological sample, comprising: contacting the sample with the antibody of claim 1; and detecting binding of the antibody to the sample, wherein a change in binding of the antibody to the sample as compared to binding of the antibody to a control sample detects α-synuclein in the biological sample.

14. An isolated nucleic acid molecule encoding the antibody of claim 1.

15. The isolated nucleic acid molecule of claim 13, wherein the nucleotide sequence encoding the antibody comprises SEQ ID NO: 1 or SEQ ID NO: 14.

16. The isolated nucleic acid molecule of claim 14, operably linked to a promoter.

17. An expression vector comprising the isolated nucleic acid molecule of claim 15.

18. An isolated host cell transformed with the expression vector of claim 16.

19. The fusion protein of claim 8, wherein the human Fc protein comprises human IgGγ Fc.

20. A chimeric antigen receptor (CAR) comprising the antibody of claim 1.

21. A bispecific antibody comprising the antibody of claim 1.

22. An isolated immunoconjugate comprising the antibody of claim 1 and a therapeutic agent.

23. The isolated immunoconjugate of claim 21, wherein the therapeutic agent comprises a drug.
